(19) United States
(12) Patent Application Publication
(10) Pub. No.: US 2023/0309839 A1
(43) Pub. Date: Oct. 5, 2023

(54) SYSTEMS AND METHODS FOR ESTIMATING CARDIAC ARRYTHMIA

(71) Applicant: CARDIOKOL LTD, Airport City (IL)

(72) Inventors: Eli ATTAR, Airport City (IL); Alon David GOREN, Airport City (IL); Amir BEKER, Airport City (IL); YIrmilyahu HAUPTMAN, Airport City (IL)

(73) Assignee: CARDIOKOL LTD, Airport City (IL)

(21) Appl. No.: 17/910,076

(22) PCT Filed: Mar. 8, 2021
(86) PCT No.: PCT/IL2021/050252

§ 371 Date: Sep. 8, 2022

Related U.S. Application Data
(60) Provisional application No. 62/987,015, filed on Mar. 9, 2020.

(51) Int. Cl. A61B 5/02; A61B 5/00; G16H 10/60; G16H 40/67; G16H 50/30; G10L 25/66; G10L 15/22; G10L 15/02

(52) U.S. Cl. CPC A61B 5/02028; A61B 5/4803; A61B 5/7278; G16H 10/60; G16H 40/67; G16H 50/30; G10L 25/66; G10L 15/22; G10L 15/02

(57) ABSTRACT

A method and system for estimating Atrial fibrillation (AF) burden based on spot checks, the method including: determining a schedule for spot checks; initiating a spot check based on the schedule; analyzing the results of the spot check to detect an AF episode; and adjusting the schedule based on detected AF episodes, where the spot check includes a type of check selected from: a voice test, an electrocardiogram (ECG) test, a photoplethysmography (PPG) test, acoustic sensing and optical heartbeat monitoring.

### FIELD OF THE INVENTION

The present invention relates generally to estimating cardiac arrhythmia of a patient and, more specifically, to adjusting a protocol of estimating cardiac arrhythmia.

### BACKGROUND OF THE INVENTION

Atrial fibrillation (AF or Afib), is one of the most common abnormal heart rhythms and is a major health problem. AF is associated with increased risk of stroke. Additionally, if a patient's heartbeat is very fast for a long period of time, it may also lead to heart failure. However, not all patients with AF are even aware of their AF situations. Some may feel uncomfortable while experiencing AF, while others don't feel AF episodes at all.

Cardiac activity of a patient, including AF, may affect the voice characteristics of the patient. For example, cardiac activity may affect the blood flow, the lungs (it is noted that the left lung shares space in the chest with the heart), the bronchi and the pleural fluid, each of which may in turn affect voice characteristics. Furthermore, heartbeat-related mechanical changes in arteries and muscles along the larynx (the “voice box”) and the vocal tract may potentially cause detectable modulations in the vocal sounds. Regardless of the exact physiological relation, the human voice is affected by the cardiac activity. Taking advantage of this relation, a cardiac condition may be detected based on analyzing a voice sample of the patient. For example, a voice sample of a patient may be used to detect an arrhythmic cardiac condition, such as atrial fibrillation (AF or Afib), e.g., as disclosed in U.S. patent application Ser. No. 16/485,173, published as US Patent Application Publication No. 2017/62457914 and entitled “Verbal periodic screening for heart disease”.

Therefore, voice analysis may provide an easy and efficient method for detecting the frequency and duration of AF condition and to evaluate the AF burden.

### SUMMARY OF THE INVENTION

According to some embodiments of the invention, a system and method for estimating AF burden based on spot checks may include determining a schedule for spot tests; initiating a spot check based on the schedule; analyzing the results of the spot check to detect an AF episode; and adjusting the schedule for next tests based on detected AF episodes.

Embodiments of the invention may include performing a plurality of spot checks according to the schedule; and estimating an AF burden based on results of the spot checks.

According to embodiments of the invention, adjusting the schedule may include adjusting at least one of the lists consisting of: frequency of performing additional spot checks, required length of the additional spot checks, and timing of performing the spot check.

According to embodiments of the invention, the spot check may include a type of check selected from: a voice test, an electrocardiogram (ECG) test, a photoplethysmography (PPG) test, acoustic sensing and optical heartbeat monitoring.

Embodiments of the invention may include selecting the type of spot check based on the detected AF episodes.

According to embodiments of the invention, the schedule may be determined based on background health and personal parameters of the patient.

According to embodiments of the invention, the schedule may be determined based on typical patterns of AF episodes.

According to embodiments of the invention, adjusting the protocol of initiating subsequent analysis may be performed based on at least one additional parameter selected from the list consisting of: patient adherence, number of previous detected AF episodes, length of previous detected AF episodes, timing of previous detected AF episodes, an AF burden frequency of past patient-initiated spot checks.

According to some embodiments of the invention, a system and method for determining a health state or a medical condition of a patient based on vocal characteristics may include obtaining a voice sample of the patient; analyzing the voice sample to determine a health state of the patient; and adjusting a protocol of initiating subsequent analysis of another voice sample of the patient based on the determined health state.

According to embodiments of the invention, adjusting the protocol of initiating subsequent analysis may include adjusting at least one of the list consisting of: frequency of taking additional voice samples of the patient, required vowels for the additional voice samples, required length of the additional voice samples, type of the additional voice samples and timing of taking the voice samples.

According to embodiments of the invention, obtaining the first voice sample of the patient may include: sampling free speech of the patient; and extracting selected vowels from the free speech to generate the first voice sample of the patient.

According to embodiments of the invention, obtaining the first voice sample of the patient may include: prompting the patient to say a predetermined set of vowels; and recording an utterance of the patient.

According to embodiments of the invention, adjusting the protocol of initiating subsequent analysis may be performed based on at least one additional parameter selected from: past determined health state, timing of previous determined health conditions, frequency of past patient-initiated analysis and measured physiological parameters.

Embodiments of the invention may include determining a required content of the subsequent voice sample based on the analysis of the first voice sample.

According to embodiments of the invention, obtaining the first voice sample of the patient and analyzing the first voice sample may be initiated by the patient.

According to embodiments of the invention, analyzing the first voice sample to determine a health state of the patient may include determining a cardiac arrhythmia of the patient.

Embodiments of the invention may include analyzing the first voice sample to determine a quality of the voice sample; and requiring an additional voice sample of the patient if the quality is below a threshold.

### BRIEF DESCRIPTION OF THE DRAWINGS

The subject matter regarded as the invention is particularly pointed out and distinctly claimed in the concluding portion of the specification. Some embodiments of the invention, however, both as to organization and method of operation, together with objects, features and advantages thereof, may best be understood by reference to the following detailed description when read with the accompanied drawings. Some embodiments of the invention are illustrated by way of example and not of limitation in the figures of the accompanying drawings, in which like reference numerals indicate corresponding, analogous or similar elements and in which:

FIG. 1 schematically illustrates a system, according to some embodiments of the invention;

FIG. 2 is a flowchart of a method for determining a health state of a patient using voice tests, according to some embodiments of the invention;

FIG. 3 is a flowchart of a method for differentiating irregular-irregularities from regular-irregularities detected in a voice sample, according to some embodiments of the invention;

FIG. 4, is a flowchart of a method for determining a health state or a medical condition of a patient using spot tests, according to some embodiments of the invention;

FIG. 5 schematically illustrates a second system, according to some embodiments of the invention; and

FIG. 6 illustrates an example computing device, according to an embodiment of the invention.

It will be appreciated that, for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

Although some embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device that manipulates and/or transforms data represented as physical (e.g., electronic) quantities within the computer's registers and/or memories into other data similarly represented as physical quantities within the computer's registers and/or memories or other information transitory or non-transitory or processor-readable storage medium that may store instructions, which when executed by the processor, cause the processor to execute operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items unless otherwise stated. Unless explicitly stated, the method embodiments described herein are not constrained to a particular order or sequence. Additionally, some of the described method embodiments or elements thereof can occur or be performed in a different order from that described, simultaneously, at the same point in time, or concurrently.

AF is typically identified by irregular heart rhythms and is clinically defined as uncoordinated contractions of the atria. AF may be asymptomatic. The presence of AF makes strokes up to five times more likely. Current medical practice manages to prevent about 60-80% of AF-related strokes. It is, therefore, a potential advantage to identify subjects suffering from AF early in order to begin medical treatment.

The durations and frequency of AF episodes may vary considerably among patients. Currently, patients are categorized as “first-detected episode of AF” for patents with a single detected AF episode, “recurrent AF” for patients with two or more AF episodes, “persistent AF” for patents with AF episodes that extend beyond 7 days, and “permanent AF” for patients with continuous AF. However, the durations and frequency of AF episodes may vary considerably among patients with recurrent and persistent AF. While some patients may experience less frequent and short episodes (e.g., length below 6-7 minutes, 6 or less times a day), others may experience more frequent and longer episodes. According to some embodiments of the invention, statistics of timing and duration of patients with similar medical diagnosis may be measured. Further, typical patterns of timing and duration of AF episodes for groups of patients (e.g., for groups of patients with similar medical diagnosis) may be generated. While the clinical definition of clinically meaningful AF patterns is vague, it may be beneficial to differentiate patients with total AF duration below 2-2.5 hours of AF a day from patients with total AF duration above 2-2.5 hours of AF a day (the exact threshold may vary among caregivers).

Some methods of detecting AF are primarily reliant on the use of continuous electrocardiogram (ECG) recordings (e.g. cardiac Holter monitors, mobile cardiac telemetry monitors, etc.). Continuous measurements are typically needed, since AF may occur only for several minutes per day and is non-symptomatic. However, continuous cardiac monitoring via ECG may present challenges, such as the precise application of a variety of electrodes, an uncomfortable apparatus, cabling, wearable sensors and issues with battery replacement or recharging. A passive monitoring has the potential advantage of identifying cardiac conditions without provoking active action steps from the screened subjects, and without having to deviate from everyday routines.

According to some embodiments of the invention, the frequency and duration of AF conditions and specifically the AF burden may be monitored and evaluated using spot checks (also referred to herein as spot tests or timed checks). Spot checks may refer to heart monitoring using any applicable manner that is not continuous and performed at selected intervals and durations. In some embodiments, a duration of a spot check may extend from few seconds to few minutes, e.g., 5 seconds to 10-20 minutes. In some embodiments, for example when extracting the heart rate from free speech, a single spot check may last as long as the patient speaks, or a few spot checks (e.g., each with a predetermined duration) may be performed as long as the patient speaks. Thus, spot checks may be less cumbersome to the patients with comparison to continuous monitoring. However, the timing and duration of spot checks need to be set so that acceptable accuracy of AF burden estimation may be achieved.

According to some embodiments of the invention, a spot test protocol, e.g., the timing and duration of spot checks, may be determined according to known typical patterns or statistics of AF episode appearances. According to some embodiments of the invention, the timing and duration of spot checks may be dynamically adjusted or tuned according to the patient condition, e.g., according to past checks and background condition of the patient. According to some embodiments of the invention, determining a spot check protocol based on known typical patterns of AF episodes, and further adjusting the protocol based on spot test results, may enable achieving a reliable estimation of when to perform spot check measurement in order to detect the AF burden. While not each and every AF episode may be detected, patients with total AF duration of above 2-2.5 hours may be differentiated with high reliability from patients with shorter total AF duration.

In some embodiments, spot checks may be initiated automatically by the system, in a dynamically scheduled configuration. Depending on the type of spot check, the user may be requested or prompted to perform the test. For example, for voice tests, the patient may be asked to speak and/or pronounce certain words or sounds. Alternatively or additionally, the spot checks may be initiated manually, by being initiated by an active action of the patient. Spot checks may include ECG, photoplethysmography (PPG), optical heartbeat monitoring, acoustic sensing (e.g., detecting acoustic signals using an acoustic sensor, and extracting the heart rate from these signals), etc. However, as disclosed herein, voice tests may provide special benefits over these technologies.

Some embodiments of the invention may provide a method for performing a voice testing, e.g., determining a health state or medical condition of a patient from a voice sample of the patient, including detecting arrhythmic cardiac condition of the patient such as AF. A voice sample of the patient may be obtained in any applicable manner, including sampling free speech or sampling an utterance of predetermined content.

Using voice testing for determining a health state or medical condition of a patient provides an easy and convenient measurement at the home setting. Any smartphone, voice service (such as Alexa®), Internet of things (IoT) devices, smart microphones, smart watch, and/or other wearable means having a microphone or an acoustic sensor, may be used for the voice sampling and no sophisticated medical device should be used. Sophisticated and cumbersome testing may not suit the home setting and may result in low levels of patient cooperation and adherence. A patient may be reluctant to wear special sensors on a daily basis for long time periods. However, as voice testing is so easy for the patient, high adherence for longer periods may be expected. Additionally, increasing the frequency of testing may not pose a serious discomfort to the patient. Furthermore, some of the voice sampling and thus the voice testing may be performed by sampling free speech, e.g., when the patient talks on the smartphone (after obtaining the patient approval), or provides instructions of a digital assistant, with no burden on the patient. This ease and comfort are extremely valuable for chronic conditions. For example, patients may live for long years with AF episodes that may come and go in changing frequencies. Thus, voice testing may be an applicable tool for home setting monitoring for AF patients.

According to some embodiments of the invention, the protocol of initiating spot tests such as ECG, PPG, an optical heartbeat monitoring, acoustic sensing, voice testing, etc., of the patient may be designed or adjusted based on the outcome of previous spot tests. The adjusted protocol parameters may include the timing, the frequency and the length of the spot test. When using voice testing, required content of the voice sample may be adjusted as well. For example, if an episode of AF is found in a spot test, the frequency of testing may increase. However, if a number of consecutive tests have shown no signs of pathology, the frequency of testing may decrease. In some embodiments, the specific statistics of a patient may be studied, e.g., the timing, frequency and duration of AF episodes of the patient may be studied using any applicable method. For example, patient statistics may be studied using continuous monitoring, using frequent spot checks or any combination thereof. The timing, frequency and length of the spot tests may be designed, tuned, or adjusted based on the AF statistics and other parameters such as the patient medical data. In some embodiments, the type of spot check to be used according to the protocol may be selected or determined based on the detected AF episodes. For example, if a less accurate type of spot check has detected an AF episode, the protocol may be adjusted to suggest using a more accurate method for subsequent spot checks.

Similarly, the timing of AF episodes (e.g., timing during the day) may affect the timing of subsequent spot tests. For example, if an AF episode is detected in the morning but not in the evening, more spot tests may be scheduled for morning hours than for the rest of the day. The frequency and timing of spot tests may be adjusted based on other parameters such as patient adherence, past determined health state, timing of previous determined health conditions, frequency of past patient-initiated analysis and other measured physiological parameters.

According to some embodiments of the invention, further testing may be performed if the quality of the sampled voice sample is not good enough (e.g., if the signal to noise ratio is not high enough), or if the results of the analysis of the voice sample, or the diagnosis, are not conclusive.

According to some embodiments of the invention, a voice sampling of the patient may be performed by a dedicated microphone, acoustic sensor or by a microphone of a smartphone or other smart device. However, if the quality of the sampled voice sample is not good enough, the patient may be requested to use a better recording device. Voice testing may be used as a first and easy screening test for cardiac conditions such as AF. If the voice test shows a probability of a cardiac condition such as AF, further examinations may be recommended by the system to be performed at the home settings or at the clinic. Home examinations may include recording of the heart rate, e.g., using for example a heart rate monitor.

According to some embodiments of the invention, voice testing may be performed in a timing and frequency to provide sufficient measurements for estimating AF burden or AF score, as disclosed herein. AF burden may refer to the amount of time that a patient's heart spends in AF over a monitoring period, although other definitions may be used. It is believed that increase in the AF burden may be associated with a higher risk of ischemic stroke and arterial thromboembolism in patients who do not receive anticoagulant medication. Thus, evaluating the AF burden may provide the physician with an effective data for evaluating the patient risk.

Some embodiments of the invention improve the technology of voice testing for detecting cardiac conditions by providing adjustability that may improve rate of detection of AF episodes and enable calculation of estimation AF burden or AF score.

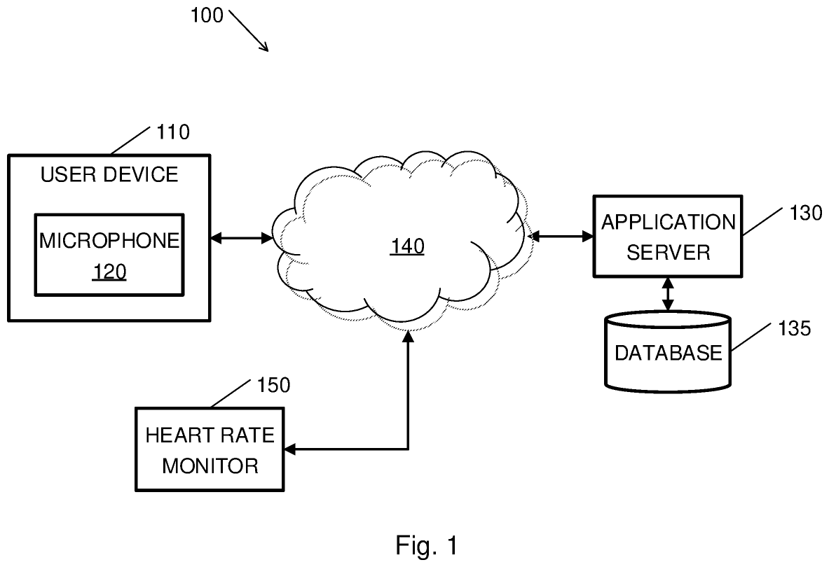

Reference is made to FIG. 1, which schematically illustrates a system 100, according to some embodiments of the invention. System 100 may include one or more user devices 110 connectable to a network 140, e.g., the internet, and optionally, heart rate monitors 150, each of which may be connectable to network 140. Each user device 110 and heart rate monitor 150 may be associated with a patient. System 100 may be configured to initiate cardiac spot tests for determining a health state of the patient, for example to detect episodes of cardiac arrhythmia such as AF. System 100 may be configured to dynamically adjust the schedule of the initiated cardiac spot tests so as to detect AF episodes, to determine or estimate a length of the detected AF episodes, and to calculate or estimate the AF burden based on the timing frequency and length of the detected AF episodes. System 100 may be configured to perfume spot checks or spot test, e.g., noncontinuous checks of the heart condition, and specifically of the heart rate to detect AF episodes. In some embodiments, the spot tests may include voice test performed by sampling speech of the patient and analyzing the speech to detect AF episodes. For example, user device 110 may include or obtain signals from microphone or acoustic sensor 120 and other hardware and software required for sampling and analyzing voice. In some embodiments, a heart monitor 150 may be used in addition or instead of the voice tests for performing the spot checks.

Heart monitor 150 may include any device used for monitoring heart rate and other heart parameters. Heart monitor 150 may sample and analyze heart signals, e.g., electrical and/or optical signals, and detect the heart rate based on those measured signals. Heart monitor 150 may be a wired or wireless device, may be or may include a wearable device. Heart monitor 150 may be or may include a smart watch, a sticker, a patch an IoT device, etc. Heart monitor 150 may be or may include an ECG device, PPG device, an optical heartbeat monitor device, or any other technology used for monitoring heart rate.

System 100, e.g., user device 110 or application server 130, may be configured to schedule spot tests for determining the heart condition of the patient. Specifically, system 100 may be configured to schedule spot tests for determining or estimating the AF burden of the patient. According to some embodiments, an initial protocol or schedule for spot checks may be determined for a patient based on background health and personal parameters of the patient such as weight, age, gender, the stroke risk of the patient, e.g., evaluated according to the CHA₂DS₂-VASc score for atrial fibrillation stroke risk, and other medical conditions. Other parameters may be considered, such as times in which cooperation of the patient is more likely, e.g., tests may be scheduled for awakening hours and not for sleeping hours. In some embodiments, system 100 may also consider the patient preferences, e.g., obtained from the patient through user device 110. The patient preferences may include time windows (e.g., hours in the day, days in the week or month, etc.) that are more convenient for him/her.

According to some embodiments, system 100 may be configured to initiate the spot test by prompting, reminding or requesting the patient to perform the spot test. Requesting or reminding the patient to perform the spot test may be performed through user device 110, e.g., by one or more of sending a test message, e.g., short message service (SMS), WhatsApp®, etc., calling the patient, ringing an alarm, etc. If a voice tests are used as spot tests, at least some of the tests may be initiated without disturbing the patient, e.g., by recording or sampling free speech. If wearable devices are used, again the spot test may be initiated and performed automatically as long as the patient wears the wearable heart monitor 150.

After one or more spot tests are performed, the schedule or protocol of the spot tests may be determined or adjusted based on, for example, the detected health state, e.g., cardiac condition, of the patient. For example, if an AF episode is detected, the frequency and length of the spot tests may be increased. The schedule or protocol of the spot tests may be determined or adjusted based on other parameters as well, for example, past determined health state, accuracy of previous spot tests, timing of previous spot tests, patient adherence to the testing protocol, timing of previous determined health conditions, frequency of past patient-initiated tests and other measured physiological parameters. For example, for AF patients, the protocol of initiating subsequent analysis may be adjusted based on at least one of: number, length and timing of previous detected AF episodes, AF burden, frequency of past patient-initiated analysis and other measured physiological parameters. Patient adherence may also affect the testing schedule. For example, more tests may be scheduled for times in the day and days in the week in which the patient is more cooperative, and fewer tests may be scheduled for times in which the patient is less cooperative. In some embodiments, the type of spot check to be used according to the protocol may be selected or determined based on the detected AF episodes. For example, if a less accurate type of spot check has detected an AF episode, the protocol may be adjusted to suggest using a more accurate method for subsequent spot checks.

In some embodiments, the spot test may include voice tests. Thus, user device 110 may record or sample voice to obtain voice samples of the patient and send the voice samples (or analyzed or partially analyzed voice samples), as well as other data, over network 140 to application server 130. User device 110 may include a communication module that may enable direct connectivity to network 140. For example, user device 110 may include a Wi-Fi or cellular module that enable direct Internet connectivity.

Application server 130 may obtain data from user device 110. Application server 130 may obtain the voice sample of the patient (or an analyzed or partially analyzed voice sample). Application server 130 may analyze the voice sample to determine a health state of the patient, for example to detect episodes of cardiac arrhythmia such as AF. Application server 130 may adjust a protocol of initiating subsequent voice tests (e.g., subsequent sampling and analysis of another voice sample) of the patient based on the determined health state. For example, application server 130 may adjust the frequency of taking additional voice samples of the patient, the length and required vowels for the additional voice samples, the type of the additional voice samples (e.g., free speech or a dictated content), timing of taking the voice samples, etc. The frequency and timing of voice testing may be adjusted based on other parameters such as past determined health state, timing of previous determined health conditions, frequency of past patient-initiated analysis and other measured physiological parameters. According to some embodiments of the invention, application server 130 may initiate further testing if the quality of the sampled voice sample is not good enough (e.g., if the signal to noise ratio is not high enough), or if the results of the analysis of the voice sample are not conclusive, e.g., if no clear diagnosis may be provided.

Application server 130 may calculate other parameters related to the health state of the patient based on the voice samples. For example, application server 130 may estimate AF burden or AF score, as disclosed herein.

Other system architecture may be used. For example, according to some embodiments, user device 110 may analyze the voice samples and adjust the examination protocol. Additionally, various data items may be provided to system 100 by other components, depending on the system design. For example, application server 130 or user device 110 may obtain patient profile and patient data from, for example, a healthcare provider, the patient himself, and/or a caregiver.

Networks 140 may include any type of network or combination of networks available for supporting communication between user device 110, application server 130, heart rate monitors 150, and databases 135. Networks 140 may include for example, wired and wireless telephone networks, the Internet and intranet networks, etc.

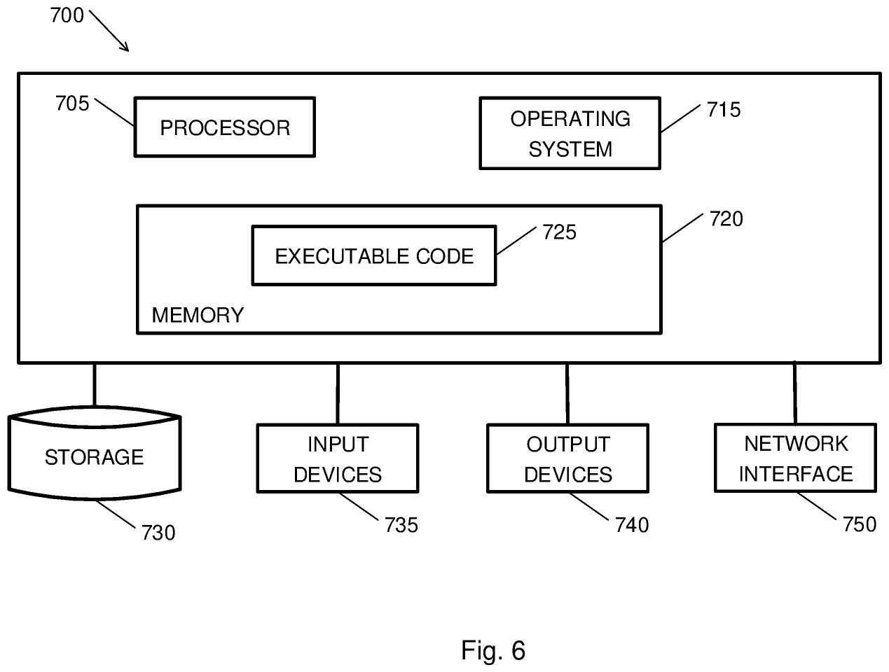

Each of user device 110, application server 130, heart rate monitors 150 may be or may include a computing device such as computing device 700 depicted in FIG. 6. One or more databases 135 may be or may include a storage device such as storage device 730. User device 110 may be or may include a smartphone, a smart microphone, a wearable microphone or acoustic sensor, a digital assistant, a smart watch, vehicle computers, fitness wearables, personal assistant computing devices, speech processing micro-controllers, monitoring bands, an Internet of Things (IoT) device, a computer or a laptop (for example, a voice sample may be recorded in a video conference such as a Zoom® meeting). User device 110 may include a microphone 120 for converting sound into an electrical signal. The electrical signal may be recorded or sampled. The recording or sampling of speech of the patient may be referred to herein as the voice sample or sampling. The voice sample may be sent to application server 130 and further processed, e.g., by user device 110 and/or application server 130, to detect a health state of the patient. Application server 130 and database 135 may be implemented in a cloud computing environment. In some embodiments user device 110 may be or may include a telephone and application server 130 may include an interactive voice response (IVR) system may call user device 110 and record the patient voice.

Analyzing a health state of a patient based on the voice sample may be performed in any applicable manner. For example, as disclosed in U.S. patent application Ser. No. 16/485,173, published as US Patent Application Publication No. 2017/62457914 and entitled “Verbal periodic screening for heart disease” which is incorporated herein in its entirety.

For example, a cardiac condition may be estimated by looking for variations over time of specific parameters that carry relevant information from the voice, for example, by analyzing voice features over time and calculating a periodicity of the values of the voice features. In some embodiments, voice features are extracted from a voice sample, optionally a spontaneous speech. In some embodiments, voice features include, for example, a weighted spectrum, and/or Linear Predictive Coefficient (LPC) and/or LPC based spectrum, and/or Mel Frequency Cepstral Coefficients (MFCC), and/or fundamental frequency (pitch), and/or energy, and/or zero crossing, and/or formants, and/or glottal pulse (vocal cord pulse), and/or jitter, and/or shimmer, and/or fractal dimension, and/or coherence, and/or wavelet analysis, or any other mathematical/statistical presentation of the speech samples. In some embodiments, analyzing the voice features may be performed using artificial intelligence (AI) or machine learning (ML) algorithms, such as deep neural networks (DNN), support vector machines (SVM), random forest etc.

In some embodiments, a heart rate of a subject is estimated, optionally by analyzing his voice sample. In some embodiments, a non-uniformity of the voice feature is used to identify irregularities in the timing of the cardiac activity, for example by identifying a periodicity at frequencies at a predetermined range around the frequency of the heart rate. In some embodiments, spectral analysis and/or autocorrelation is used to identify periodic and/or semi-periodic changes in the voice sample. In some embodiments, periodicity is calculated in a band width of a spectral peak at the predetermined range of the heart rate, of a voice feature. Typically, the wider the band width, the lower the periodicity, and therefore the higher the probability for an arrhythmia. In some embodiments, in order to determine the cardiac condition, the band width is compared to a predetermined threshold.

In some embodiments, a characterizing parameter of the periodicity is compared to a threshold to determine the cardiac condition. For example, a peak of an autocorrelation function (of a voice feature, such as pitch) around the frequency of the heart rate may be characterized by its band width, and a band width of the autocorrelation function having a value above a predetermined threshold would be associated with a high probability for an arrhythmic cardiac condition.

In some embodiments, spectral cross-coherence of the speech is calculated between segments of the speech, optionally around the pitch and\or formant frequencies and\or around any frequencies that are potentially affected by the heart pulse. Coherence reaching lower values for a short period of time can be an indication of heart pulse. In this manner, heart pulses can be located on the speech time line.

In some embodiments, the distribution of the values of the voice feature is determined, for example the standard deviation. In some embodiments, a characterizing parameter of the shape of the distribution is compared to a threshold to determine the cardiac condition. For example, a large width of the shape of the distribution, and/or of the spectral peak values, could be compared to a predetermined threshold which is associated with a high probability for an arrhythmic cardiac condition.

In some embodiments, a multi-feature classifier is optionally used (combining several features) and an optionally multi-dimensional threshold over the multi-dimensional distribution of the values of the voice features is determined, for example using a SVM method, and/or Vector Quantization methods such as K-MEANS clustering analysis and DNN.

In some embodiments, a characterizing parameter of the shape of the multi-dimensional distribution is compared to a multi-dimensional threshold to determine the cardiac condition.

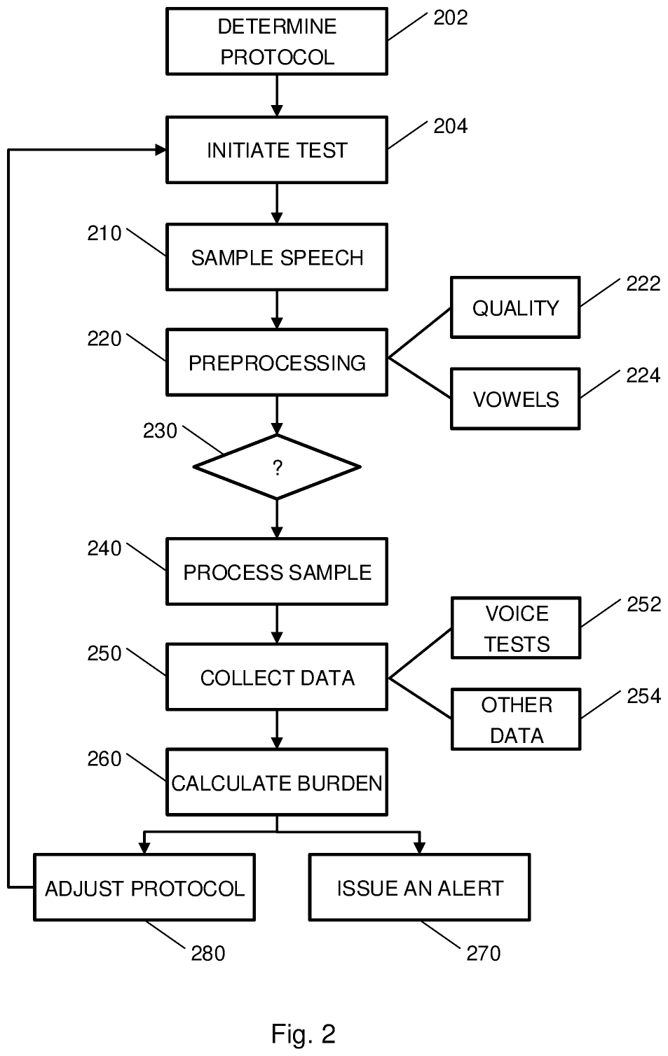

Reference is made to FIG. 2, which is a flowchart of a method for determining a health state or a medical condition of a patient using voice tests, according to some embodiments of the invention. An embodiment of a method for determining a health state or medical condition of a patient may be performed, for example, by the systems shown in FIGS. 1, 5 and 6, although other hardware may be used.

In operation 202 an initial protocol or schedule for voice tests may be determined. According to some embodiments, the initial protocol or schedule may be determined based on background health and personal parameters of the patient such as weight, sleeping hours, general health condition and other sickness, environmental conditions, age, gender, the stroke risk of the patient, e.g., evaluated according to the CHA₂DS₂-VASc score for atrial fibrillation stroke risk, and other medical conditions. Furthermore, the initial protocol or schedule may be determined based on known typical patterns of AF episodes, e.g., associated with patients having the same background health and personal parameters. Other parameters may be considered, such as times in which cooperation of the patient is more likely, e.g., tests may be scheduled for awakening hours and not for sleeping hours, patient preferences obtained from the patient e.g., time windows (e.g., hours in the day, days in the week or month, etc.) that are more convenient for the patient.

In operation 204, a voice test or voice check may be initiated. For example, the patient may be prompted or requested to pronounce vowels for a voice test. In some embodiments, a voice test may be initiated automatically. Additionally or alternatively, other spot tests, such as ECG or PPG, may be initiated for testing the heart condition, evaluating the heart rate and the presence of an AF episode and other pathologies. In some embodiments, a multiple types of spot checks may be initiated to increase the number of spot checks to increase the reliability of analysis.

The voice test may be initiated by requesting or prompting the patient to perform the test, e.g., to pronounce vowels, e.g., using user device 110. The voice tests may be initiated based on the determined protocol or schedule for voice tests. Additionally or alternatively, spot tests may be initiated by detecting and recording free or spontaneous speech or in response to a request of the patient. It should be noted, however, that the voice tests that are initiated, if free or spontaneous speech is detected or in response to a request of the patient, may be performed in addition to the scheduled checks, or instead of the scheduled tests if voice is detected or a request form the patient for a voice test is obtained in temporal proximity to the scheduled test.

In operation 210, speech of a patient may be sampled or recorded. Speech may be sampled in any applicable manner, using a microphone and a recorder of any applicable device, e.g., by user device 110. For example, using a smartphone, a smart watch, vehicle computers, fitness wearables, personal assistance computing devices, speech processing micro-controllers, monitoring bands, a computer or a laptop. According to some embodiments, the user may be prompted to say a predetermined set of vowels that are required for the speech processing algorithm. For example, a required content of the voice sample may be presented to the patient, and an utterance of the patient may be sampled. The required vowels may include ‘ahh’, ‘ehhh’, ‘eee’, required words may include asking the patient to count from one to ten, or say a sentence like “The current time is five twenty”. Additionally or alternatively, free speech may be sampled. The test may be initiated manually, by a human caller from a call center, or automatically using recorded voice or machine speech locally or from a remote location, the test may be initiated by the mobile device automatically, according to a pre-set or dynamic protocol. As disclosed herein, the protocol may be dynamically adjusted based on test results or diagnoses. In some embodiments, the patient may initiate voice test in addition to or instead of the system-initiated tests.

In operation 220 preprocessing may be performed. According to some embodiments, preprocessing may be performed by the recording device, e.g., user device 110. According to some embodiments, the sampled speech may be sent to another device for preprocessing, e.g., to application server 130. According to some embodiments, preprocessing may include determining the quality of the sampled speech, as indicated in block 222. For example, the signal to noise ratio (SNR) of the sampled speech, pitch stability, microphone saturation, or other quality measurements may be calculated. According to some embodiments, selected vowels may be extracted from a voice recording in order to generate the voice sample of the patient, as indicated in operation 224. In some embodiments, operation 224 may be applied to free speech recordings or sample in order to extract from the free speech vowels that are required for the speech processing algorithm.

In operation 230, it may be determined whether the quality of the voice sample is good enough. If the quality of the voice sample is good enough, e.g., above a threshold, the voice sample may be further processed as indicated in block 240, otherwise the method may return to operation 210 to sample more speech. Additionally or alternatively, it may be determined whether enough vowels were extracted in operation 224. Determining whether enough vowels were sampled may be beneficial for free speech sampling. For example, AF detection may require a predetermined set of vowels, e.g., type of vowels and quantity of each vowel. For example, AF detection may require a recording of the vowels ahh′, ‘ehhh’, ‘eee’ several times, each time for a few seconds. When sampling free speech, there is no way to guarantee that enough vowels are present in the speech sample. Thus, in operation 230, the type and quantity of vowels that were extracted from the free speech may be compared with the number and type of vowels required for performing the voice analysis. If enough vowels were sampled, the voice sample may be further processed as indicated in block 240, otherwise the method may return to operation 210 to sample more speech. In some embodiments, only if the quality of the voice recording is good enough, e.g., above a threshold, and enough vowels were sampled will the voice sample be further processed as indicated in block 240, otherwise the method may return to operation 210 to sample more speech.

In operation 240, the voice sample may be processed or analyzed to determine a health state or a medical condition of the patient. According to some embodiments, processing may be performed by the recording device, e.g., user device 110. According to some embodiments, the sampled speech or the pre-processed speech may be sent to another device, e.g., to application server 130, for processing. According to some embodiments, processing may include extracting at least one voice feature from the voice sample (or preprocessed voice sample) and determining a health state or a medical condition of the patient based on the voice features. According to some embodiments, a cardiac arrhythmia, e.g., an AF episode, may be detected by processing the voice sample. According to some embodiments of the invention, heart rate irregularities may be detected, and irregular-irregularities may be differentiated from regular-irregularities, as disclosed herein.

In operation 242, it may be determined whether the results of the processing are conclusive, e.g., if a confidence level of the results is above a threshold. If the results are conclusive, the method may proceed to operation 250. If the results are inconclusive, then the method may return to operation 210 in order to sample more speech.

In operation 250, data regarding the health state or a medical condition of the patient may be collected. In some embodiments, results of a plurality of voice tests, e.g., each of which is an outcome of operation 240, may be collected, as indicated in block 252. According to some embodiments, data from other sources may be obtained, as indicated in clock 254. For example, information may be obtained from a healthcare provider (e.g., a doctor or a nurse). Additionally or alternatively, the patient may use other medical devices to measure physiological parameters such as heart rate, ECG, etc., and those measured physiological parameters may be provided in operation 254.

In operation 260, a general health condition of the patient may be estimated, based on the collected data. For example, for patients with AF, AF burden, also referred to as AF score, may be estimated. AF burden may be defined as the total duration of the detected AF episodes in a time period (e.g., in 24 or 48 hours), the duration of the longest detected AF episode in a time period, number of AF episodes in a time period, or the percentage of time the patient is in AF during a certain monitoring period, etc.

AF burden is typically defined by total duration of the detected AF episodes in a time period:

$$A {F}_{burden} = \frac{Σ duration of AF episodes}{time period} .$$

However, other definitions of AF burden may be used. Some examples for alternative ways for calculating AF burden are provided below. Let E denote the number of detected AF episodes in a time period, e.g., a day, a week, 10 days, a month, let AAF denote the average length of AF episodes detected during the time period, let MAF denote the median length of detected AF episodes during the time period, and let pAF90 denote a length of detected AF episode separating the top 10 percent longest AF episodes from shorter AF episodes, detected during the time period. AF may be calculated using any of the equations presented in table 1, or a combination thereof.

TABLE 1
Equations for calculating AF burden.

| | | |
|---|---|---|
| AF_{burden} = E * AAF | AF_{burden} = E * MAF | AF_{burden} = E * pAF90 |
| AF_{burden} = log(E) * AAF | AF_{burden} = log(E) * MAF | AF_{burden} = log(E) * pAF90 |
| AF_{burden} = E * log(AAF) | AF_{burden} = E * log(MAF) | AF_{burden} = E * log(pAF90) |
| AF_{burden} = AAF \hat{ } (log(E)) | AF_{burden} = MAF \hat{ } (log(E)) | AF_{burden} = pAF90 \hat{ } (log(E)) |

In operation 270, an alert may be issued. The alert may be issued in case an AF episode is detected or in case the AF burden is above a threshold. The alert may be provided in the form of a text message, an audible alarm or any other manner. The alert may be provided to the user (e.g., through user device 130, or to the healthcare provider. Operation 270 may include providing a report of the performed tests, including timing of test, patient adherence, test results, and the calculated AF burden.

In operation 280, the protocol for initiating subsequent voice tests may be adjusted based on the determined health state, and the method may return to operation 204 for initiating subsequent tests. For example, for AF patients, the protocol of initiating subsequent analysis may be adjusted based on at least one of: number, length and timing of previous detected AF episodes, the AF burden, frequency of past patient-initiated analysis and other measured physiological parameters such as heart rate of the patient measured by a heart rate measurement device. In some embodiments, initiating subsequent spot test may include initiating more voice tests. Additionally or alternatively, initiating subsequent spot test may include initiating other types of tests, e.g., ECG or PPG, for validation of the voice test. For example, the protocol of initiating subsequent voice tests may include adjusting at least one of: frequency of taking additional voice samples of the patient, required vowels for the additional voice samples, required length of the additional voice samples, type of the additional voice samples (e.g., free speech or predetermined vowels) and timing of taking the voice samples. According to some embodiments of the invention, adjusting the protocol of initiating subsequent voice test may be performed based on at least one additional parameter such as: past determined health state, patient adherence to the testing protocol, timing of previous determined health conditions, frequency of past patient-initiated analysis and other measured physiological parameters. According to some embodiments, required content of the voice sample may be determined based on a previous analysis of a previous voice sample.

According to some embodiments the protocol of initiating subsequent analysis may be adjusted according to patient adherence or compliance. For example, if a patient performs the required tests in certain times and does not perform tests in other times, more tests may be determined to the times in the day or the day in the week in which the patient is cooperative and performs the required tests. Similarly, less frequent tests may be scheduled for patients that do not adhere to the protocol, in an effort to increase patient adherence. Less frequent tests may, however, increase the time required to obtain an estimation of the AF burden. On the other hand, more frequent tests may be scheduled to the more cooperative patients, e.g., patients that adhere well to the testing protocol, reducing the time required to calculate the AF burden.

In an example embodiment, an initial voice testing protocol requires testing the patient a few times a day. If an AF episode is detected, the frequency of testing may increase. If no episodes are detected for a few days, the number of voice tests may decrease to the initial value. For example, the initial voice testing protocol may require testing the patient twice a day. If an AF episode is detected, the frequency of testing may increase by one to four tests a day. If no episodes are detected for three days, the number of voice tests may decrease to the initial value.

In an example embodiment, the initial protocol requires sampling three categories of vowels and text for each voice test. If for example, an AF episode is detected more frequently in a certain category, more tests may be initiated in this category.

In an example embodiment, the initial protocol requires testing the patient twice a day, one time at 8:00 AM and one time at 8:00 PM. If, for example, an AF episode is detected in the 8:00 PM test, an additional test or tests may be added in the evening, one, two or three hours before or after the 8:00 PM test.

In an example embodiment, voice testing and heart rate measurements are performed. If, for example, AF episodes were detected concurrently or in close proximity to irregular heart rate as detected by a heart rate monitor, a voice testing may be initiated if irregular heart rate is detected by the heart rate monitor.

In an example embodiment, an initial protocol may test free speech only, e.g., voice recorded or sampled while the patient talks on the phone or gives voice commands. If an AF episode is detected in at least one of those tests, the system may initiate more tests. For example, the system may initiate a test every 10 minutes following the positive results (a detected AF episode) until at least two tests provide negative results (no detected AF episode). After at least two tests with negative results, the frequency of the system-initiated tests may decrease gradually.

In operation 280, test results or diagnosis may be reported to the patient and/or to the healthcare provider. The report may include the time of test and test results or diagnosis. The report may further include an alert in case a heart condition such as AF is detected.

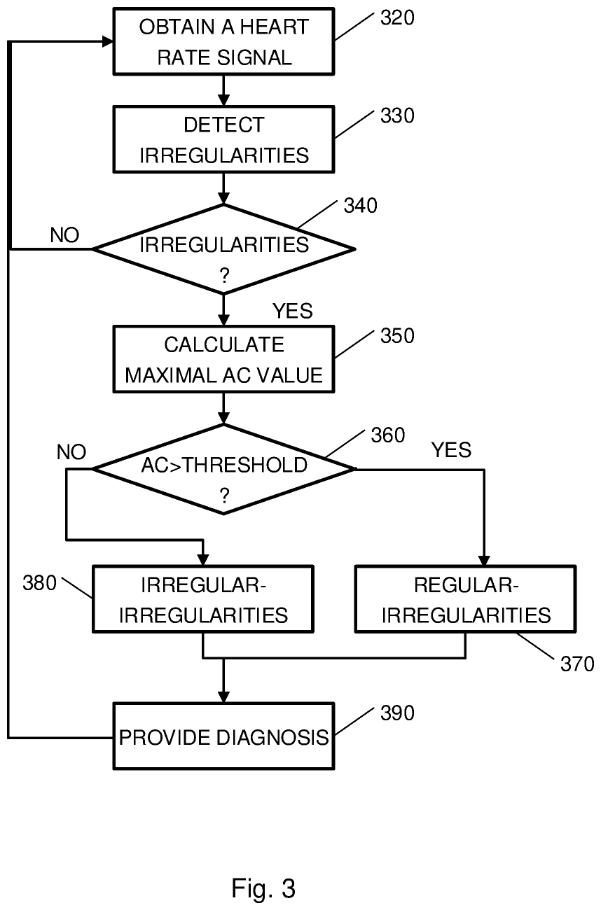

Reference is made to FIG. 3, which is a flowchart of a method for differentiating irregular-irregularities from regular-irregularities detected in a voice sample, according to some embodiments of the invention. An embodiment of a method for differentiating irregular-irregularities from regular-irregularities detected in a voice sample may be an elaboration of operation 240 depicted in FIG. 2. An embodiment of a method for differentiating irregular-irregularities from regular-irregularities detected in a voice sample may be performed, for example, by the systems shown in FIGS. 1, 5 and 6, although other hardware may be used.

Heart rate irregularities may be divided into regular-irregularities and irregular-irregularities. This division may have clinical significance since each type of irregularity may be a result of different pathologies. For example, regular-irregularities may be a result of ventricular or super ventricular ectopic activity, and irregular-irregularities may be a result of multiple ectopic beats, AF or atrial flutter. In many applications, it may be beneficial to differentiate regular-irregularities from irregular-irregularities. The classification of irregularities into regular-irregularities and irregular-irregularities may provide a significant diagnostic value to the healthcare provider.

In operation 320, a heart rate (HR) signal or function may be obtained. In some embodiments, a heart rate function may be generated, estimated or calculated based on a voice sample, an ECG signal, a PPG signal, an optical heartbeat monitoring signal, an acoustic heartbeat monitoring signal, etc. An instantaneous HR signal may be calculated for each cardiac cycle as the reciprocal of the interval between successive beats. The instantaneous HR signal may be resampled to generate the HR signal, denoted Y. The time intervals for sampling the instantaneous HR signal may be either constant time intervals or may equal the heart-beat intervals.

In operation 330, heart rate irregularities may be detected from the heart rate signal, e.g., from a part or portion of the heat rate signal having a time duration or a number of samples. If irregularities are not detected, then, as indicated in operation 340, some embodiments of the method may proceed to analyze new hear rate signal. If, however, irregularities are detected, then some embodiments of the method may proceed to calculate a maximal discrete autocorrelation (AC) value, as indicated in operation 350. In operation 350, an AC function, rₖ, may be calculated according to:

$${r}_{k} = \frac{{\sum }_{i = 1}^{N - k} ( {Y}_{i} - \overline{Y} ) ( {Y}_{i + k} - \overline{Y} )}{{Σ}_{i = 1}^{N} {( {Y}_{i} - \overline{Y} )}^{2}}$$

Where k denotes step or shift size, N denotes a length of the heart rate signal (e.g., in samples), Y denotes the heart rate signal, and Y̅ denotes average heart rate in the tested heart rate signal.

A maximal AC value may equal max (rₖ). In operation 360, it may be determined whether the maximal AC value is above a threshold. If the maximal AC value is above a threshold, then the irregularity may be defined as regular irregularity, as indicated in operation 370. If, however, the maximal AC value is not above a threshold (e.g., equal or below the threshold), then the irregularity may be defined as irregular irregularity, as indicated in operation 380. In operation 390, the diagnosis may be provided to the user, e.g., the patient or the healthcare provider, and the method may proceed to operation 310 to obtain another voice sample.

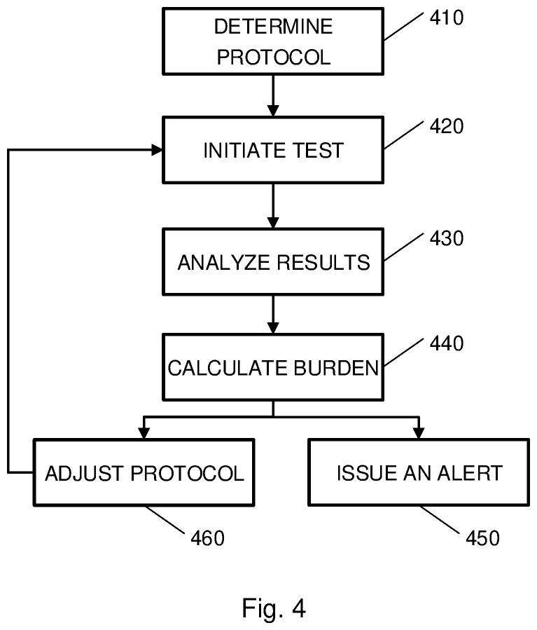

Reference is made to FIG. 4, which is a flowchart of a method for determining a health state or a medical condition of a patient using spot tests, according to some embodiments of the invention. An embodiment of a method for determining a health state or medical condition of a patient using spot tests may be performed, for example, by the systems shown in FIGS. 1, 5 and 6, although other hardware may be used.

In operation 410, an initial protocol or schedule for spot tests may be determined. According to some embodiments, the initial protocol or schedule may be determined based on background health and personal parameters of the patient such as weight, age, gender, the stroke risk of the patient, e.g., evaluated according to the CHA₂DS₂-VASc score for atrial fibrillation stroke risk, and other medical conditions. Furthermore, the initial protocol or schedule may be determined based on known typical patterns of AF episodes, e.g., associated with patients having the same background health and personal parameters. Other parameters may be considered, such as times in which cooperation of the patient is more likely, e.g., tests may be scheduled for awakening hours and not for sleeping hours, patient preferences obtained from the patient e.g., time windows (e.g., hours in the day, days in the week or month, etc.) that are more convenient for the patient.

In operation 420, a spot test or spot check may be initiated. The spot test may include any applicable heart rate monitoring technique, such as voice test, ECG, PPG, optical heartbeat monitoring, acoustic sensing, that is not performed continuously. The spot test may be initiated by requesting or prompting the patient to perform the test, e.g., to apply heart monitor or heart rate monitor 150. In operation 430, the results of the spot test may be analyzed to determine if a cardiac pathology, e.g., AF episode is detected. According to some embodiments, the result of the present test may be analyzed together with results of previous tests. According to some embodiments, data from other sources may be obtained. For example, information may be obtained from a healthcare provider.

In operation 440, a general health condition of the patient may be estimated, based on the collected data. For example, for patients with AF, AF burden, also referred to as AF score, may be estimated. AF burden may be defined as the total time the patient is in AF during a certain monitoring period, the duration of the longest detected AF episode, number of AF episodes, or the percentage of time that the patient is in AF during a certain monitoring period, etc., similarly to operation 260.

In operation 450, an alert may be issued. The alert may be issued in case an AF episode is detected or in case the AF burden is above a threshold, similarly to operation 270. In operation 460, the protocol of initiating subsequent spot tests may be adjusted based on the detected AF episodes, and the method may return to operation 410 for initiating subsequent tests. For example, the protocol of initiating subsequent analysis may be adjusted based on at least one of: number, length and timing of previous detected AF episodes, the AF burden, frequency of past patient-initiated spot checks and other measured physiological parameters such as heart rate of the patient measured by a heart rate measurement device. For example, the protocol of initiating subsequent spot tests may include adjusting at least one of: frequency of performing additional spot tests, required length of the additional spot tests, and timing of performing the spot tests. According to some embodiments of the invention, adjusting the protocol of initiating subsequent spot tests may be performed based on at least one additional parameter such as: patient adherence, past determined health state, patient adherence to the testing protocol, timing of previous determined health conditions, frequency of past patient-initiated analysis and other measured physiological parameters.

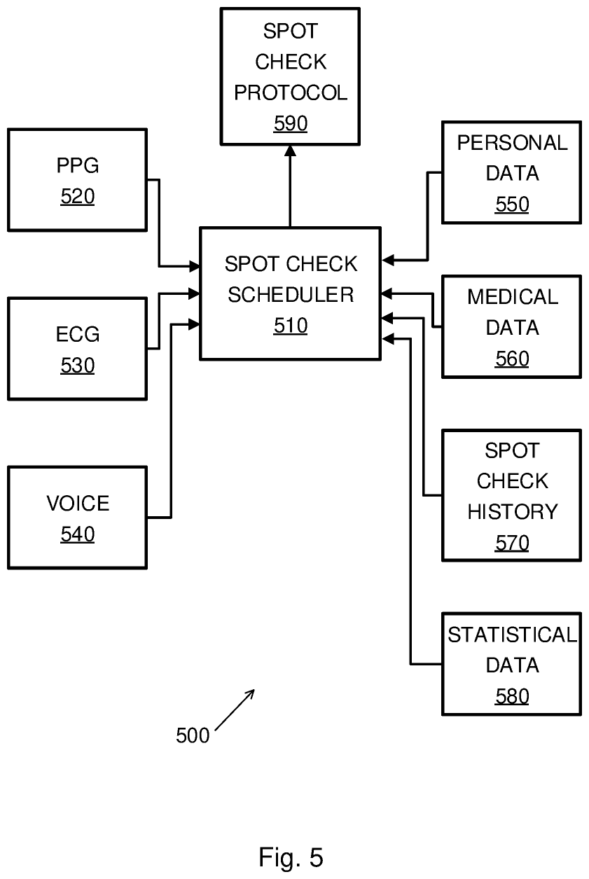

Reference is made to FIG. 5, which schematically illustrates a system 500, according to some embodiments of the invention. System 500 may include a spot check scheduler 510, configured to determine and update or adjust a schedule or protocol for spot checks for a patient according to embodiments of the invention. Spot check scheduler 510 may be implemented, for example, in user device 110, application server 130 or computing device 700, or a combination thereof. Other hardware may be used. Spot check scheduler 510 may obtain spot check data, e.g., spot check results or spot check sample or measurements from which spot check results may be determined. For example, spot check scheduler 510 may obtain a voice sample 540, generated by any applicable voice sampling device such as, but no limited to, a smartphone, a smart microphone, a wearable microphone or acoustic sensor, a digital assistant, a smart watch, vehicle computers, fitness wearables, personal assistant computing devices, speech processing micro-controllers, monitoring bands, an Internet of Things (IoT) device, a computer or a laptop (for example, a voice sample may be recorded in a video conference such as a Zoom® meeting). In some embodiments, spot check scheduler 510 may analyze the voice sample to determine a health state of the patient, e.g., to detect an AF episode or to determine if the voice sample indicates presence of an AF episode. In some embodiments, spot check scheduler 510 may obtain the analysis results, e.g., a determination of an AF episode, or chances of an AF episode, based on a voice sample.

Spot check scheduler 510 may obtain spot check data from other devices, or using other methods. For example, Spot check scheduler 510 may obtain spot check data from PPG device 520, ECG device 530, and/or other devices. Spot check scheduler 510 may obtain raw data and analyze the raw data to detect AF episodes, or obtain analyzed data, e.g., a determination of an AF episode, or chances of an AF episode, based on PPG, ECG, etc.

Spot check scheduler 510 may obtain background data such as personal data 550, medical data 560, spot checks history 570 and statistical data 580. Personal data 550 may include for example personal parameters of the patient such as weight, age, gender, and other personal parameters. Medical data 560 may include medical conditions of the patient such as the stroke risk, background diseases, prescribed medications, and other medical conditions. Spot checks history 570 may include data related to previous spot checks performed by system 500 and/or system 100 to the patient. The data may include the type of spot check performed, the timing of the spot check, patient adherence to the scheduled spot checks (e.g., data regarding scheduled spot checks that were not performed by the patient may be also stored and available to spot check scheduler 510) and the results of the spot checks, e.g., determination of presence/non-presence of AF and or chances for presence/non-presence of AF and confidence level. Statistical data 580 may include statistics of spot checks history 570, e.g., average time and duration of previously detected AF episodes, AF burden, etc.

Spot check scheduler 510 may integrate data obtained from the data sources to determine a schedule or protocol for future or subsequent spot checks and/or to adjust the schedule or protocol based on detected AF episodes and other data, as disclosed herein. Spot check scheduler 510 may determine a timing and type of a next spot check, and may initiate the next spot check. For example, spot check scheduler 510 may activate the appropriate device to obtain the determined spot check at the determined time, and may notify the patient of the schedule or protocol for subsequent spot checks. Spot check scheduler 510 may alert the patient when the time to perform a spot check arrives.

FIG. 6 illustrates an example computing device 700 according to an embodiment of the invention. Various components such as user device 110, heart rate monitor 150, application server 130, spot check scheduler 510, and other modules, may be or may include computing device 700, or may include components such as shown in FIG. 6. For example, a first computing device 700 with a first processor 705 may be used to determine a health state of a patient.

Computing device 700 may include a processor 705 that may be, for example, a central processing unit processor (CPU), a chip or any suitable computing or computational device, an operating system 715, a memory 720, a storage 730, input devices 735 and output devices 740. Processor 705 may be or include one or more processors, etc., co-located or distributed. Computing device 700 may be for example a workstation or personal computer or may be at least partially implemented by one or more remote servers (e.g., in the “cloud”).

Operating system 715 may be or may include any code segment designed and/or configured to perform tasks involving coordination, scheduling, arbitration, supervising, controlling or otherwise managing operation of computing device 700, for example. Operating system 715 may be a commercial operating system. Memory 720 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 720 may be or may include a plurality of, possibly different memory units.

Executable code 725 may be any executable code, e.g., an application, a program, a process, task or script. Executable code 725 may be executed by processor 705 possibly under control of operating system 715. For example, executable code 725 may be or include an application to determining a health state of a patient. In some embodiments, more than one computing device 700 may be used. For example, a plurality of computing devices that include components similar to those included in computing device 700 may be connected to a network and used as a system.

Storage 730 may be or may include, for example, a hard disk drive, a floppy disk drive, a Compact Disk (CD) drive, a CD-Recordable (CD-R) drive, a universal serial bus (USB) device or other suitable removable and/or fixed storage unit. In some embodiments, some of the components shown in FIG. 6 may be omitted. For example, memory 720 may be a non-volatile memory having the storage capacity of storage 730. Accordingly, although shown as a separate component, storage 730 may be embedded or included in memory 720.

Input devices 735 may be or may include a mouse, a keyboard, a touch screen or pad or any suitable input device. It will be recognized that any suitable number of input devices may be operatively connected to computing device 700 as shown by block 735. Output devices 740 may include one or more displays, speakers and/or any other suitable output devices. It will be recognized that any suitable number of output devices may be operatively connected to computing device 700 as shown by block 740. Any applicable input/output (I/O) devices may be connected to computing device 700 as shown by blocks 735 and 740. For example, a wired or wireless network interface card (NIC), a modem, printer or facsimile machine, a universal serial bus (USB) device or external hard drive may be included in input devices 735 and/or output devices 740. Network interface 750 may enable device 700 to communicate with one or more other computers or networks. For example, network interface 750 may include a Wi-Fi or Bluetooth device or connection, a connection to an intranet or the internet, an antenna etc.

Some embodiments described in this disclosure may include the use of a special purpose or general-purpose computer including various computer hardware or software modules, as discussed in greater detail below.

Some embodiments within the scope of this disclosure also include computer-readable media, or non-transitory computer storage medium, for carrying or having computer-executable instructions or data structures stored thereon. The instructions, when executed, may cause the processor to carry out some embodiments of the invention. Such computer-readable media, or computer storage medium, can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the term “module” or “component” can refer to software objects or routines that execute on the computing system. The different components, modules, engines, and services described herein may be implemented as objects or processes that execute on the computing system (e.g., as separate threads). While the system and methods described herein are preferably implemented in software, implementations in hardware or a combination of software and hardware are also possible and contemplated. In this description, a “computer” may be any computing system as previously defined herein, or any module or combination of modulates running on a computing system.

For the processes and/or methods disclosed, the functions performed in the processes and methods may be implemented in differing order as may be indicated by context. Furthermore, the outlined steps and operations are only provided as examples, and some of the steps and operations may be optional, combined into fewer steps and operations, or expanded into additional steps and operations.

The present disclosure is not to be limited in terms of the particular embodiments described in this application, which are intended as illustrations of various aspects. Many modifications and variations can be made without departing from its scope. Functionally equivalent methods and apparatuses within the scope of the disclosure, in addition to those enumerated, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used in this disclosure is for the purpose of describing particular embodiments only and is not intended to be limiting.

This disclosure may sometimes illustrate different components contained within, or connected with, different other components. Such depicted architectures are merely exemplary, and many other architectures can be implemented which achieve the same or similar functionality.

Aspects of the present disclosure may be embodied in other forms without departing from its spirit or essential characteristics. The described aspects are to be considered in all respects illustrative and not restrictive. The claimed subject matter is indicated by the appended claims rather than by the foregoing description. All changes which come within the meaning and range of equivalency of the claims are to be embraced within their scope.

1. A method for estimating Atrial fibrillation (AF) burden based on spot checks, the method comprising:
determining a schedule for spot checks;
initiating a spot check based on the schedule;
analyzing the results of the spot check to detect an AF episode; and
adjusting the schedule based on detected AF episodes.

2. (canceled)

3. The method of claim 1, wherein adjusting the schedule comprises adjusting at least one of the lists consisting of: frequency of performing additional spot checks, required length of the additional spot checks, and timing of performing the spot check.

4. The method of claim 1, wherein the spot check comprises a type of check selected from the list consisting of: a voice test, an electrocardiogram (ECG) test, a photoplethysmography (PPG) test, acoustic sensing and optical heartbeat monitoring, and wherein the method comprising selecting the type of spot check based on the detected AF episodes.

5. (canceled)

6. The method of claim 1, wherein the schedule is determined based on background health and personal parameters of the patient.

7. The method of claim 1, wherein the schedule is determined based on typical patterns of AF episodes.

8-17. (canceled)

18. A device for estimating Atrial fibrillation (AF) burden based on spot checks, the device comprising:
a memory; and
a processor configured to:
determine a schedule for spot checks;
initiate a spot check based on the schedule;
analyze the results of the spot check to detect an AF episode; and
adjust the schedule based on detected AF episodes.

19. The device of claim 18, wherein the processor is configured to:
perform a plurality of spot checks according to the schedule; and
estimate an AF burden based on results of the spot checks.

20. The device of claim 18, wherein the processor is configured to adjust the schedule by adjusting at least one of the lists consisting of: frequency of performing additional spot checks, required length of the additional spot checks, and timing of performing the spot check.

21. The device of claim 18, wherein the spot check comprises a type of check selected from the list consisting of: a voice test, an electrocardiogram (ECG) test, a photoplethysmography (PPG) test, acoustic sensing and optical heartbeat monitoring.

22. The device of claim 21, wherein the processor is configured to select the type of spot check based on the detected AF episodes.

23. The device of claim 18, wherein the processor is configured to determine the schedule based on background health and personal parameters of the patient.

24. The device of claim 18, wherein the processor is configured to determine the schedule based on typical patterns of AF episodes.

25. The device of claim 18, wherein the processor is configured to adjust the protocol of initiating subsequent analysis based on at least one additional parameter selected from the list consisting of: patient adherence, number of previous detected AF episodes, length of previous detected AF episodes, timing of previous detected AF episodes, an AF burden frequency of past patient-initiated spot checks.

26. A device for determining a health state of a patient, the device comprising:
a memory; and
a processor configured to:
obtain a first voice sample of the patient;
analyze the first voice sample to determine a health state of the patient; and
adjust a protocol of initiating subsequent analysis of a subsequent voice sample of the patient based on the determined health state.

27. The device of claim 26, the processor is configured to adjust the protocol of initiating subsequent analysis by adjusting at least one of the list consisting of: frequency of taking additional voice samples of the patient, required vowels for the additional voice samples, required length of the additional voice samples, type of the additional voice samples and timing of taking the voice samples.

28. The device of claim 26, wherein the processor is configured to obtain the first voice sample of the patient by:
sampling free speech of the patient; and
extracting selected vowels from the free speech to generate the first voice sample of the patient.

29. The device of claim 26, wherein the processor is configured to obtain the first voice sample of the patient by:
prompting the patient to say a predetermined set of vowels; and
recording an utterance of the patient.

30. (canceled)

31. The device of claim 26, wherein the processor is configured to determine a required content of the subsequent voice sample based on the analysis of the first voice sample.

32. The device of claim 26, wherein obtaining the first voice sample of the patient and analyzing the first voice sample is initiated by the patient.

33. The device of claim 26, wherein the processor is configured to analyze the first voice sample to determine a health state of the patient by determining a cardiac arrhythmia of the patient.

34. (canceled)
